(19) United States
(12) Patent Application Publication
(10) Pub. No.: US 2025/0221717 A1
(43) Pub. Date: Jul. 10, 2025

(54) DEVICE FOR PRESSING BODY PART

(71) Applicant: AIRS MEDICAL INC., Seoul (KR)

(72) Inventor: Changkyun KIM, Gyeonggi-do (KR)

(21) Appl. No.: 19/090,402

(22) Filed: Mar. 26, 2025

Related U.S. Application Data
(63) Continuation of application No. PCT/KR2023/008549, filed on Jun. 20, 2023.

(30) Foreign Application Priority Data
Oct. 13, 2022 (KR) 10-2022-0131394

(51) Int. Cl. A61B 17/132; A61B 17/00

(52) U.S. Cl. CPC A61B 17/1322; A61B 2017/00398; A61B 2017/00477; A61B 2017/00862

(57) ABSTRACT

Disclosed herein is a device for compressing a body part according to one embodiment of the present disclosure. The device may include: a compression unit including an elastic member configured to come into contact along the circumference of a body part; a power transmission unit including a first power transmission module coupled with an actuator and configured to perform rotational movement; and a connection unit provided at one end of the power transmission unit and configured to enable the attachment and detachment of the elastic member. In this case, the compression unit may apply a constant pressure along the circumference of the body part by the first power transmission module designed in a spiral shape even when the circumference of the body part changes during a process of compressing the body part.

### CROSS-REFERENCE TO RELATED APPLICATION

This application is a Bypass Continuation Application of PCT/KR2023/008549 filed on Jun. 20, 2023, which claims priority to Korean Patent Application No. 10-2022-0131394 filed on Oct. 13, 2022. The entire disclosure of each of the foregoing applications is incorporated herein by reference in its entirety.

### TECHNICAL FIELD

The content of the present disclosure relates to a device for compressing a body part, and more particularly, to a device capable of applying a constant pressure to an overall body part regardless of the circumference of the body part on which a medical procedure is performed.

### BACKGROUND ART

In order for medical professionals to perform medical procedures, the tasks of compressing body parts may first be performed. For example, when blood is collected from a vein, a medical professional may first compress the upper arm and then bring a needle close to the brachial or lower arm. In this case, the compression of the upper arm is performed for the purposes of venous congestion and accurate injection through vascular dilatation.

According to clinical studies on the relationship among upper arm compression pressure, the time of compression and the cross-sectional area of the vein, it is recommended that upper arm compression for venous blood collection be performed by applying a pressure of 60 mmHg for 30 to 60 seconds. In other words, it can be seen that for effective venous blood collection, it is necessary to continuously apply a constant pressure to the upper arm.

Since the circumference of the upper arm may change depending on the compression, a band-type device in which the change in the circumference of the upper arm affects the pressure may not continuously apply a constant pressure to the upper arm. Accordingly, one solution may be to adjust the pressure applied by the band while measuring the pressure via a pressure or load sensor. However, this approach has disadvantages in that the structure of a device must become complicated in order to utilize a sensor and, as the structure becomes more complicated, it becomes difficult to perform the replacement of a component attributable to contamination. Furthermore, this approach has a disadvantage in that a considerable additional cost is required to construct the complicated structure.

### DISCLOSURE

### Technical Problem

The present disclosure has been conceived in response to the above-described background art, and an object of the present disclosure is to provide a device that may apply a constant pressure to a body part regardless of changes in the circumference of the body part without a separate sensor.

In addition, an object of the present disclosure is to provide a device for compressing a body part that enables the easy replacement of a component even when contamination occurs during a medical procedure such as blood collection.

However, the objects to be achieved in the present disclosure are not limited to the objects mentioned above, and other objects not mentioned may be clearly understood based on the following description.

### Technical Solution

According to one embodiment of the present disclosure for achieving the above-described object, there is disclosed a device for compressing a body part. The device may include: a compression unit including an elastic member configured to come into contact along the circumference of a body part; a power transmission unit including a first power transmission module coupled with an actuator and configured to perform rotational movement; and a connection unit provided at one end of the power transmission unit and configured to enable the attachment and detachment of the elastic member. The compression unit may apply a constant pressure along the circumference of the body part by the first power transmission module designed in a spiral shape even when the circumference of the body part changes during a process of compressing the body part.

Alternatively, the power transmission unit may further include a second power transmission module connected to one end of the first power transmission module and configured to convert the rotational movement into linear movement.

Alternatively, one end of the second power transmission module may be connected to one end of the first power transmission module that is most adjacent to the actuator, and the other end of the second power transmission module may be coupled to the connection unit.

Alternatively, the radius of the first power transmission module may be designed based on the mathematical relationship between the pressure applied to the body part and the tension of the elastic member so that the tension of the elastic member generated by the rotation of the first power transmission module corresponds to the difference between the initial length of the elastic member and the moving distance of the second power transmission module.

Alternatively, the elastic member may form a loop in which the body part is accommodated in such a manner that the one and other ends thereof are attached to the connection unit.

Alternatively, the compression unit may further include one or more rollers configured to guide the direction of tension of the elastic member to one side by gathering the elastic member that surrounds the body part.

Alternatively, the compression unit may further include a guide member configured to prevent the elastic member from moving away from an original position during a process in which the elastic member is moved by the tension.

Alternatively, the radius of the first power transmission module may be determined based on the stall torque of the actuator, the initial length of the elastic member, and a polar coordinate representation for the shape of the first power transmission module.

Alternatively, the radius of the first power transmission module may be designed based on the following mathematical relationship so that a constant pressure is applied along the circumference of the body part:

$$R ( θ ) = \frac{1}{2} \sqrt{\frac{1}{\frac{2 A}{{τ}_{max}} θ + \frac{{A}^{2} {L}_{0}^{2}}{{τ}_{max}^{2}}}}$$

where R is the radius, τₘₐₓ is the stall torque of the actuator, L₀ is the initial length of the elastic member, and θ is the polar coordinate representation for the shape of the first power transmission module.

### Advantageous Effects

The device according to the present disclosure may apply a constant pressure to a body part regardless of changes in the circumference of the body part without a separate sensor.

In addition, the device according to the present disclosure enables the easy replacement of a component even when contamination occurs during a medical procedure such as blood collection.

### DESCRIPTION OF DRAWINGS

FIG. 1 is a conceptual diagram illustrating a conventional tourniquet;

FIG. 2 is a block diagram of a device according to one embodiment of the present disclosure;

FIG. 3 is a plan view of a device according to one embodiment of the present disclosure; and

FIG. 4 is a conceptual diagram illustrating the detailed configuration of a device according to one embodiment of the present disclosure.

### MODE FOR INVENTION

Embodiments of the present disclosure will be described in detail below with reference to the accompanying drawings so that those having ordinary skill in the art of the present disclosure (hereinafter those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “N-th (N is a natural number)” used herein may be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

The term “connected” used herein should be interpreted to include not only a case where components are “directly connected” to each other but also a case where another component is “present” therebetween and a case where they are “electrically connected” to each other with another component interposed therebetween.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The “medical robot” of the present disclosure may be a general name for robots that support or assist all medical procedures performed at medical sites. For example, the “medical robot” may include a venipuncture robot that includes a blood collection or intravenous injection (IV) function for the diagnosis of diseases, a blood transfusion, and/or the like. In this case, the “medical robot” may include an end-effector and parts necessary for blood collection or intravenous injection.

The foregoing descriptions of the terms are intended to help to understand the present disclosure. Accordingly, it should be noted that unless the above-described terms are explicitly described as limiting the content of the present disclosure, they are not used in the sense of limiting the technical spirit of the present disclosure.

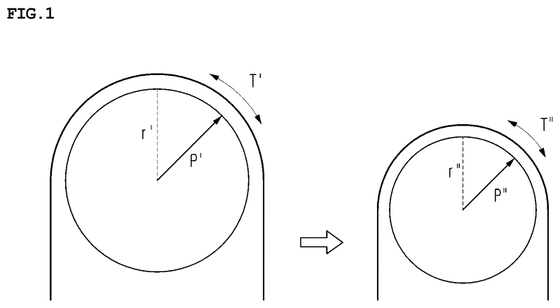

FIG. 1 is a conceptual diagram illustrating a conventional tourniquet.

The conventional tourniquet of FIG. 1 illustrates a band-type device that compresses a body part such as the upper arm to perform a medical procedure such as blood collection. The conventional tourniquet assists a medical procedure such as blood collection by compressing a body part such as the upper arm by using the tension of a band. For example, when the upper arm is accommodated inside a loop formed by the band as shown in the left view of FIG. 1, the conventional tourniquet compresses the upper arm based on the tension of the band. In this case, the pressure P′ applied to the upper arm may be represented by tension T′/(radius r′ of the upper arm*width t′ of the band). Here, the friction between the surface of the upper arm and the band is not taken into consideration, and the pressure inside the upper arm is assumed to be uniform. Furthermore, the upper arm is assumed to be a circle having a constant radius. As for the mathematical relationship of P′ described above, it can be seen that the pressure applied to the upper arm by the conventional tourniquet is affected by the circumference of the upper arm.

Meanwhile, the circumference and size of a body part may change due to external pressure. For example, the circumference of the upper arm may change by nearly two times depending on the compression. According to clinical research results, when the basic circumference of the upper arm is 31 cm, the circumference of the upper arm may be reduced up to 16 cm depending on the compression. Accordingly, referring to FIG. 1, when the upper arm is compressed by the band, its circumference may change from that of the left view to that of the right view of FIG. 1. That is, the radius of the upper arm may be reduced from r′ to r″, which is smaller than r′. Accordingly, even when the tension of the band and the width of the band are the same, the radius of the upper arm changes during a compression process, so that the pressure applied to the upper arm cannot help but change. When the circumference of the upper arm decreases due to the compression, the pressure applied by the conventional tourniquet cannot help but change from P′ to P″. That is, in the case of the conventional tourniquet, during a compression process, a constant pressure may not be applied to the upper arm due to the influence of changes in the circumference of the upper arm.

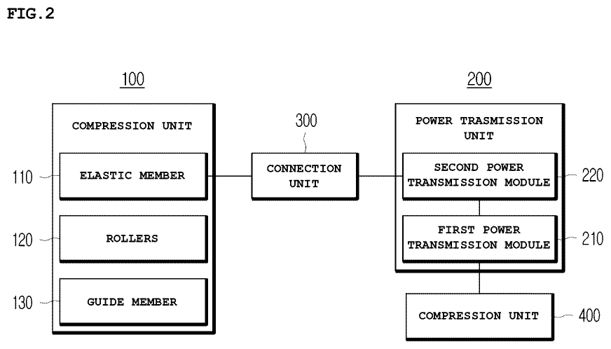

FIG. 2 is a block diagram of a device according to one embodiment of the present disclosure.

The device according to the one embodiment of the present disclosure may be an independent device for assisting a medical procedure, or may be one component of a medical robot. Referring to FIG. 2, the device according to the one embodiment of the present disclosure may include a compression unit 100 configured to restrict the flow of blood by compressing a body part on which a medical procedure is performed, a power transmission unit 200 configured to control an operation for implementing the compression function of the compression unit 100, a connection unit 300 configured to connect the compression unit 100 and the power transmission unit 200, and an actuator 400 configured to generate power for implementing the compression function of the compression unit 100.

The compression unit 100 may include an elastic member 110 configured to come into contact along the circumference of a body part. The elastic member 110 may perform a function of directly coming into contact with a body part to be compressed and compressing the body part. The elastic member 110 may be pulled by the tension generated via the power transmission unit 200 and compress the body part along the overall circumference thereof. For example, the elastic member 110 may come into contact with the upper arm for blood collection and compress the upper arm by using the tension generated via the power transmission unit 200. In this case, the elastic member 110 may be in the form of a band made of a chemical material that has elasticity and can adhere to the skin. The elastic member 110 may restrict the flow of blood to facilitate blood collection by coming into close contact with the upper arm and compressing the upper arm in all directions.

The compression unit 100 may further include one or more rollers 120 configured to assist in the movement of the elastic member 110. The rollers 120 may form a path for the elastic member 110 that is moved by the tension generated via the power transmission unit 200. The rollers 120 may be disposed adjacent to the elastic member 110 and perform pivoting so that the elastic member 110 can easily extend or contract along the direction of the tension generated via the power transmission unit 200. That is, the rollers 120 may be disposed adjacent to the elastic member 110 and perform pivoting, thereby minimizing friction with other components during the process in which the elastic member 110 extends or contracts. Furthermore, the rollers 120 may include a plurality of rollers and be disposed along the path along which the elastic member 110 is moved.

In addition, the compression unit 100 may further include a guide member 130 configured to assist in the movement of the elastic member 110. The guide member 130 may form a path for the elastic member 110 that is moved by the tension generated via the power transmission unit 200, together with the rollers 120. That is, the guide member 130 may function as an inner wall that allows the elastic member 110 to move without moving away from an initially disposed position. When tension is generated in the elastic member 110 via the power transmission unit 200, the elastic member 110 may extend or contract along the path formed by the guide member 130. Accordingly, the guide member 130 may prevent the elastic member 110 from moving away from an initially disposed position (i.e. an original position) during a process of extending or contracting.

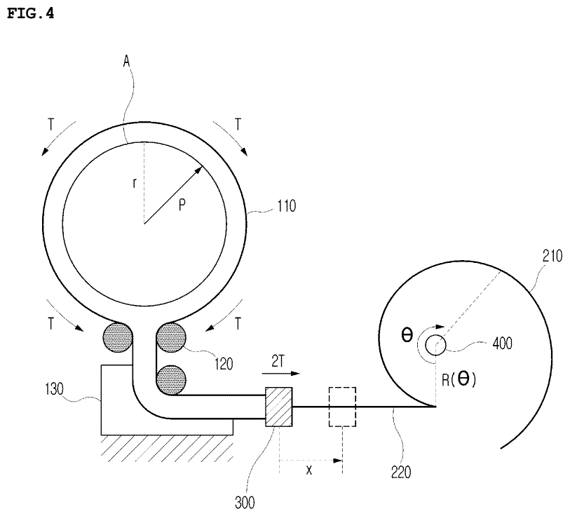

The power transmission unit 200 may include a first power transmission module 210 coupled with the actuator 400 and configured to perform rotational movement. The first power transmission module 210 may perform pivoting by means of the power generated by the actuator 400, and may generate the tension of the elastic member 110, used to compress a body part, through the pivoting. In this case, in order to allow a constant pressure to be continuously applied to a body part regardless of the circumference of the body part that changes as the compression unit 100 performs compression, the first power transmission module 210 may be a spiral rotation body. Furthermore, the spiral rotation body may be made of sheet metal. That is, the compression unit 100 may apply a constant pressure along the circumference of a body part by means of the first power transmission module 210 designed in a spiral shape even when the circumference of the body part changes during a process of compressing the body part. The relationship between the spiral first power transmission module 210 and the pressure will be specifically described through FIG. 4 to be described below.

The power transmission unit 200 may include a second power transmission module 220 configured to convert the rotational movement of the first power transmission module 210 into linear movement. The second power transmission module 220 may be connected to the first power transmission module 210 and convert the pivoting movement of the first power transmission module 210 into linear movement, and may generate the tension of the elastic member 110, used to compress a body part, through the linear movement. That is, the second power transmission module 220 may serve as an intermediate medium that transmits the power, transmitted via the rotating first power transmission module 210, to the elastic member 110. In this case, the second power transmission module 220 may be made of sheet metal.

The connection unit 300 may connect one end of the power transmission unit 200 and the ends of the elastic member 110. In this case, the one end of the power transmission unit 200 may be fixed to the connection unit 300, but the elastic member 110 may be selectively attached to and detached from the connection unit 300. More specifically, the connection unit 300 may connect one end of the second power transmission module 220 and the ends of the elastic member 110. In this case, the elastic member 110 may be mounted on or removed from the connection unit 300 by a user. When the elastic member 110 is coupled to the connection unit 300, power is transmitted to the elastic member 110 from the actuator 400 through the first power transmission module 210 and the second power transmission module 220, and thus, tension may be generated in the elastic member 110. When the elastic member 110 is separated from the connection unit 300, power may not be transmitted to the elastic member 110 from the actuator 400 through the first power transmission module 210 and the second power transmission module 220. When the elastic member 110 is configured to be coupled to or separated from the connection unit 300 in this manner, a user may easily replace the elastic member 110 when the elastic member 110 is contaminated during a medical procedure such as blood collection.

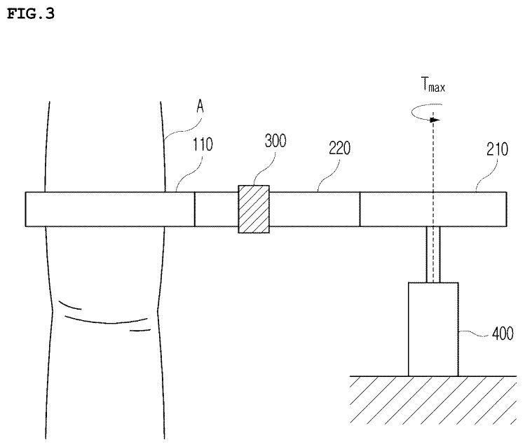

FIG. 3 is a plan view of a device according to one embodiment of the present disclosure. Furthermore, FIG. 4 is a conceptual diagram illustrating the detailed configuration of a device according to one embodiment of the present disclosure.

Referring to FIGS. 3 and 4, the elastic member 110 may form a loop that comes into close contact along the circumference of a body part A. The elastic member 110 may form a loop in which the body part A is accommodated in such a manner that one end and the other end of the elastic member 110 are coupled to the connection unit 300. When the elastic member 110 forms a loop, the body part A may be accommodated in the inner space of the loop and comes into contact with the inner side of the elastic member 110. For example, when the upper arm needs to be compressed for blood collection, the upper arm may be accommodated in the inner space of the loop formed by the elastic member 110. Furthermore, when the elastic member 110 is extended by tension, pressure may be applied to the upper arm in close contact with the inner surface of the elastic member 110. In this case, the elastic member 110 forms a loop that surrounds the overall circumference of the upper arm, and thus, may compress the upper arm in all directions, unlike the conventional tourniquet of FIG. 1.

The rollers 120 may guide the direction of tension of the elastic member 110 to one side by gathering the elastic member 110 that surrounds the body part A. The rollers 120 are disposed at the contact points where the elastic member 110 gathers while forming a loop, so that the elastic member 110 can be gathered to one side. Since the connection unit 300, the first power transmission module 210, and the second power transmission module 220 are sequentially connected along the direction in which the elastic member 110 is gathered through the rollers 120, the direction of tension of the elastic member 110 may correspond to the direction in which the elastic member 110 is gathered via the rollers 120. The elastic member 110 gathered to the locations where the rollers 120 are disposed may naturally extend or contract along the pivotable rollers 120 when tension is generated.

The rollers 120 may be placed at corners of the path, along which the elastic member 110 is extended or contracted, for the smooth movement of the elastic member 110. The rollers 120 may be placed at respective corners where the elastic member 110 is bent, and may determine the path of the elastic member 110. Furthermore, the rollers 120 may be placed at respective corners where the elastic member 110 is bent, and may minimize friction that may occur as the elastic member 110 moves at the bending points. The elastic member 110 may implement stable and smooth movement via the rollers 120 placed at the point where the elastic member 110 is gathered and corner points, thereby compressing the overall upper arm.

The guide member 130 may be formed in the same shape as the path, along which the elastic member 110 moves, in order to prevent the elastic member 110 from deviating from the path. For example, at the point where the elastic member 110 is bent to the right, a pivotable roller 120 may be placed at the inner corner point where friction occurs a lot due to tension, as shown in FIG. 4. Furthermore, at the point where the elastic member 110 is bent to the right, the guide member 130 formed in a shape to guide movement upward or to the right may be placed at the outer corner point where friction occurs relatively little but there is a high possibility of deviating, as shown in FIG. 4. In this case, when the first power transmission module 210 rotates in the first direction, the elastic member 110 may be extended along the path formed by the rollers 120 and the guide member 130 in the direction in which the tension is guided via the rollers 120. When the first power transmission module 210 rotates in a second direction opposite to the first direction, the elastic member 110 may be contracted along the path formed by the rollers 120 and the guide member 130 and return to an initial state. In this manner, the rollers 120 and the guide member 130 may be disposed on the path for the movement of the elastic member 110 according to their respective purposes.

One end of the second power transmission module 220 may be connected to one end of the first power transmission module 210 that is closest to the actuator 400. Furthermore, the other end of the second power transmission module 220 may be coupled to the connection unit 300. More specifically, one end of the second power transmission module 220 may be connected to the starting point of the first power transmission module 210 that is closest to the rod of the actuator 400 that becomes the central axis of the first power transmission module 210. Furthermore, the other end of the second power transmission module 220 may be connected to the connection unit 330 to which the elastic member 110 is detachably attached.

The first power transmission module 210 may be directly connected to the actuator 400 and rotate. The first power transmission module 210 may have a spiral structure having a radius R (0) based on the center of the rod of the actuator 400, which is the center of rotation. The elastic member 110 may perform compression with a constant pressure along the circumference of the body part A by means of the first power transmission module 210 having the above spiral structure.

More specifically, as for the relationship between the first power transmission module 210 having a spiral structure and the pressure applied to the body part A, the pressure P applied to the body part A and the circumference C of the body part A may be represented as follows:

$$P = \frac{T}{rt} \qquad ( 1 )$$

$$C = {L}_{0} - 2 x + \frac{T}{k} = 2 π r , r = \frac{{L}_{0} - 2 x + \frac{T}{k}}{2 π} \qquad ( 2 )$$

T denotes the tension of the elastic member 110, r denotes the radius of the body part A, t denotes the width of the elastic member 110, L₀ denotes the initial length of the elastic member 110 (i.e. the length in the original state where no tension is applied), x denotes the distance by which the second power transmission module 220 is moved according to the rotation of the first power transmission module 210 (i.e., the distance by which the elastic member 110 is pulled), and k denotes the elastic coefficient of the elastic member 110. In this case, the friction between the surface of the body part A and the elastic member 110 is not taken into consideration, and the pressure inside the body part A is assumed to be uniform. Furthermore, the body part A is assumed to be a circle having a constant radius.

When the pressure P applied to the body part A is rearranged by considering the relationship between Equations 1 and 2, the following Equation 3 may be derived, as follows:

$$P = \frac{2 π}{( \frac{{L}_{0} - 2 x}{T} + \frac{1}{k} ) t} \qquad ( 3 )$$

In this case, when the tension T satisfies T=A (L₀−2x), the pressure P becomes a constant, so that the pressure P applied to the body part A can be constant regardless of the radius r. In this case, A denotes a design constant. That is, when the tension T of the elastic member 110 generated by the rotation of the first power transmission module 210 corresponds to the difference between the initial length L₀ of the elastic member 110 and the moving distance x of the second power transmission module 220, the pressure P applied to the body part A may be constant regardless of the value of the radius r of the body part A. Accordingly, the radius R(θ) of the first power transmission module 210 may be designed based on the mathematical relationship between the pressure P applied to the body part A and the tension T of the elastic member 110 so that the tension T of the elastic member 110 corresponds to the difference between the initial length L₀ of the elastic member 110 and the moving distance x of the second power transmission module 220.

Since the tension T corresponds to a value obtained by dividing the stall torque τₘₐₓ of the actuator 400 by 2*R(θ), which is the diameter of the first power transmission module 210, R(θ) may be represented by Equation 4 below based on the condition of T=A(L₀−2x) described above:

$$R ( θ ) = \frac{{τ}_{max}}{2 A ( {L}_{0} - 2 x )} \qquad ( 4 )$$

In this case, the moving distance x of the second power transmission module 220 corresponds to ∫₀^{θ}R(θ)dθ, so that Equation 4 can be converted into Equation 5 below:

$$R ( θ ) = \frac{1}{2} \sqrt{\frac{1}{\frac{2 A}{{τ}_{max}} θ + \frac{{A}^{2} {L}_{0}^{2}}{{τ}_{max}^{2}}}} \qquad ( 5 )$$

That is, it can be seen that when the radius of the first power transmission module 210 is designed based on Equation 5 above, the pressure P applied to the body part A becomes a constant and has a constant value regardless of the radius r of the body part A.

In other words, the radius R(θ) of the first power transmission module 210 may be designed to satisfy the relationship of the above-described Equation 5 based on the stall torque τₘₐₓ of the actuator 400, the initial length of the elastic member 110, and a polar coordinate representation θ for the helical shape of the first power transmission module 210. When the first power transmission module 210 is designed in a helical shape to satisfy this mathematical relationship, a constant pressure may be applied along the overall circumference of the body part A regardless of changes in the circumference even when the circumference of the body part A changes during a compression process.

In summary, the device according to one embodiment of the present disclosure may apply a constant pressure to a body part regardless of the circumference of the body part via the mathematically designed spiral first power transmission module 210. Since the device according to one embodiment of the present disclosure does not require a separate sensor for maintaining the pressure, the function of applying constant compression may be easily implemented at a low cost. Furthermore, the device according to one embodiment of the present disclosure enables the easy replacement of a component (e.g., the elastic member 110 or the like) that comes into contact with the body part A even when contamination such as blood splashing occurs during a medical procedure such as blood collection, via the connection unit 300.

The various embodiments of the present disclosure described above may be combined with one or more additional embodiments, and may be changed within the range understandable to those skilled in the art in light of the above detailed description. The embodiments of the present disclosure should be understood as illustrative but not restrictive in all respects. For example, individual components described as unitary may be implemented in a distributed manner, and similarly, the components described as distributed may also be implemented in a combined form. Accordingly, all changes or modifications derived from the meanings and scopes of the claims of the present disclosure and their equivalents should be included in the scope of the present construed as being disclosure.

1. A device for compressing a body part, the device comprising:
a compression unit including an elastic member configured to come into contact along a circumference of a body part;
a power transmission unit including a first power transmission module coupled with an actuator and configured to perform rotational movement; and
a connection unit provided at one end of the power transmission unit and configured to enable attachment and detachment of the elastic member;
wherein the compression unit applies a constant pressure along the circumference of the body part by the first power transmission module designed in a spiral shape even when the circumference of the body part changes during a process of compressing the body part.

2. The device of claim 1, wherein the power transmission unit further comprises a second power transmission module connected to one end of the first power transmission module and configured to convert the rotational movement into linear movement.

3. The device of claim 2, wherein:
one end of the second power transmission module is connected to one end of the first power transmission module that is most adjacent to the actuator; and
a remaining end of the second power transmission module is coupled to the connection unit.

4. The device of claim 2, wherein a radius of the first power transmission module is designed based on a mathematical relationship between a pressure applied to the body part and a tension of the elastic member so that the tension of the elastic member generated by rotation of the first power transmission module corresponds to a difference between an initial length of the elastic member and a moving distance of the second power transmission module.

5. The device of claim 1, wherein the elastic member forms a loop in which the body part is accommodated in such a manner that the one and remaining ends thereof are attached to the connection unit.

6. The device of claim 1, wherein the compression unit further comprises one or more rollers configured to guide a direction of tension of the elastic member to one side by gathering the elastic member that surrounds the body part.

7. The device of claim 1, wherein the compression unit further comprises a guide member configured to prevent the elastic member from moving away from an original position during a process in which the elastic member is moved by the tension.

8. The device of claim 1, wherein a radius of the first power transmission module is determined based on a stall torque of the actuator, an initial length of the elastic member, and a polar coordinate representation for a shape of the first power transmission module.

9. The device of claim 8, wherein the radius of the first power transmission module is designed based on the following mathematical relationship so that a constant pressure is applied along the circumference of the body part:
$$R ( θ ) = \frac{1}{2} \sqrt{\frac{1}{\frac{2 A}{{τ}_{max}} θ + \frac{{A}^{2} {L}_{0}^{2}}{{τ}_{max}^{2}}}}$$
where R is the radius, τₘₐₓ is the stall torque of the actuator, L₀ is the initial length of the elastic member, and θ is the polar coordinate representation for the shape of the first power transmission module.
